(19) United States
(12) Patent Application Publication
(10) Pub. No.: US 2024/0108302 A1
(43) Pub. Date: Apr. 4, 2024

(54) METHOD FOR IDENTIFYING INTERVENTIONAL OBJECT, IMAGING SYSTEM, AND NON-TRANSITORY COMPUTER-READABLE MEDIUM

(71) Applicant: GE Precision Healthcare LLC, Waukesha, WI (US)

(72) Inventors: Xiaodong Lu, Beijing (CN); Yingqing Lyu, Beijing (CN); Jingyuan Dong, Beijing (CN)

(21) Appl. No.: 18/477,218

(22) Filed: Sep. 28, 2023

(30) Foreign Application Priority Data
Sep. 30, 2022 (CN) 202211217560.3

(51) Int. Cl. A61B 6/00; A61B 6/03

(52) U.S. Cl. CPC A61B 6/547; A61B 6/032; A61B 6/463

(57) ABSTRACT

Provided in the present application is a method for identifying an interventional object, including acquiring volumetric data regarding a subject to be scanned, and generating a first volumetric image on the basis of the volumetric data, acquiring position information of the interventional object relative to the subject to be scanned, determining a second volumetric image on the basis of the position information, the second volumetric image having a range smaller than the first volumetric image, and identifying the interventional object in the second volumetric image. Further provided in the present application are an imaging system and a non-transitory computer-readable medium.

### CROSS-REFERENCE TO RELATED APPLICATIONS

This application claims priority to Chinese Application No. 202211217560.3, filed on Sep. 30, 2022, the disclosure of which is incorporated herein by reference in its entirety.

### TECHNICAL FIELD

The present application relates to the field of medical imaging, and in particular to a method for identifying an interventional object, an imaging system, and a non-transitory computer-readable medium.

### BACKGROUND

Interventional procedures are a conventional medical means. In some application scenarios, a medical object is punctured by an interventional object (e.g., a needle). After the interventional object is manipulated into a predetermined position (e.g., a lesion), operations such as sampling and drug administration can be performed. In the above process, imaging of the interventional object and the medical object is important for precise puncturing. Computed tomography (CT) is one of the imaging techniques used in the interventional procedures. Using the CT imaging technique, the position of the interventional object in the interior of the body of a subject to be scanned can be promptly grasped during performance of the interventional procedure, thereby guiding the operation of the interventional procedure.

It is of great significance to accurately identify the interventional object in a generated CT volumetric image. For example, the identification of the interventional object is a basis for tracking thereof. After accurately identifying the interventional object in the volumetric image, a CT imaging system can continuously update the position of the interventional object during the interventional procedure so as to perform tracking. For another example, the CT imaging system can adjust parameters, directions, etc., of the volumetric image after identifying the interventional object, which facilitates viewing of the interventional object by an operator. However, the identification of the interventional object in the CT volumetric image is easily interfered with by other objects such as bones. In addition, the efficiency of identifying a small interventional object in an image within a large volume range is usually limited. Accurate and quick identification of interventional objects remains a challenge.

### SUMMARY

The aforementioned defects, deficiencies, and problems are solved herein, and said problems and solutions will be understood through reading and understanding the following description.

In some embodiments of the present application, a method for identifying an interventional object is provided. The method includes acquiring volumetric data regarding a subject to be scanned, and generating a first volumetric image on the basis of the volumetric data; acquiring position information of the interventional object relative to the subject to be scanned, determining a second volumetric image on the basis of the position information, the second volumetric image having a range smaller than the first volumetric image, and identifying the interventional object in the second volumetric image.

In some embodiments of the present application, an imaging system is provided. The imaging system includes a volumetric data acquisition apparatus, for acquiring volumetric data regarding a subject to be scanned, a processor. The processor is configured to acquire the volumetric data regarding the subject to be scanned and generate a first volumetric image on the basis of the volumetric data, acquire position information of an interventional object relative to the subject to be scanned, determine a second volumetric image on the basis of the position information, the second volumetric image having a range smaller than the first volumetric image, and identify the interventional object in the second volumetric image; and a display, for receiving a signal from the processor so as to carry out display.

In some embodiments of the present application, a non-transitory computer-readable medium is further provided. The non-transitory computer-readable medium has a computer program stored thereon, which has at least one code segment executable by a machine so as to enable the machine to perform the following steps of acquiring volumetric data regarding a subject to be scanned, and generating a first volumetric image on the basis of the volumetric data; acquiring position information of an interventional object relative to the subject to be scanned, determining a second volumetric image on the basis of the position information, the second volumetric image having a range smaller than the first volumetric image, and identifying the interventional object in the second volumetric image.

It should be understood that the brief description above is provided to introduce, in a simplified form, concepts that will be further described in the detailed description. However, the brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, wherein:

FIG. 1 is a perspective view of an imaging system in some embodiments of the present application;

FIG. 2 is a schematic block diagram of an imaging system in some embodiments of the present application;

FIG. 3 is a flowchart of a method for identifying an interventional object in some embodiments of the present application;

FIG. 4 is a schematic diagram of determining a position range of an interventional object in a first volumetric image in some embodiments of the present application;

FIG. 5 is a flowchart of a method for identifying an interventional object in some other embodiments of the present application; and

FIG. 6 is a schematic diagram of identifying an interventional object in some embodiments of the present application.

### DETAILED DESCRIPTION

Specific embodiments of the present application are described below. It should be noted that in the specific description of said embodiments, for a concise description, the present application may not describe in detail all of the features of the actual embodiments. It should be understood that in the actual implementation process of any embodiment, just as in the process of any one engineering project or design project, a variety of specific decisions are often made to achieve specific goals of a developer and to meet system-related or business-related constraints, which may also vary from one embodiment to another. Furthermore, it should also be understood that although efforts made in such development processes may be complex and extended, for a person of ordinary skill in the art related to the disclosure of the present application, some design, manufacturing or production changes made on the basis of the technical disclosure of the present disclosure are only conventional technical means, and it should not be construed that the content of the present disclosure is insufficient.

Unless otherwise defined, the technical or scientific terms used in the claims and the description should be as the terms are usually understood by those possessing ordinary skill in the technical field to which the present invention pertains. The terms “first”, “second” and similar words used in the present application and the claims do not express any order, quantity or importance, but are merely intended to distinguish between different constituents. The terms “one” or “a/an” and similar terms do not express a limitation on quantity, but rather that at least one is present. The terms “include” or “comprise” and similar words indicate that an element or object preceding the terms “include” or “comprise” encompasses elements or objects and equivalent elements thereof listed after the terms “include” or “comprise,” and do not exclude other elements or objects. The terms “connect” or “link” and similar words are not limited to physical or mechanical connections, and are not limited to direct or indirect connections.

In addition, while a CT system is described in the present application by way of example, it should be understood that the present technology may also be useful when applied to images acquired by using other imaging modalities, such as an X-ray imaging system, a magnetic resonance imaging (MRI) system, a positron emission tomography (PET) imaging system, a single photon emission computed tomography (SPECT) imaging system, and combinations thereof (e.g., a multi-modal imaging system such as a PET/CT, PET/MR, or SPECT/CT imaging system). The discussion of the CT imaging system in the present invention is provided only as an example of one suitable imaging system.

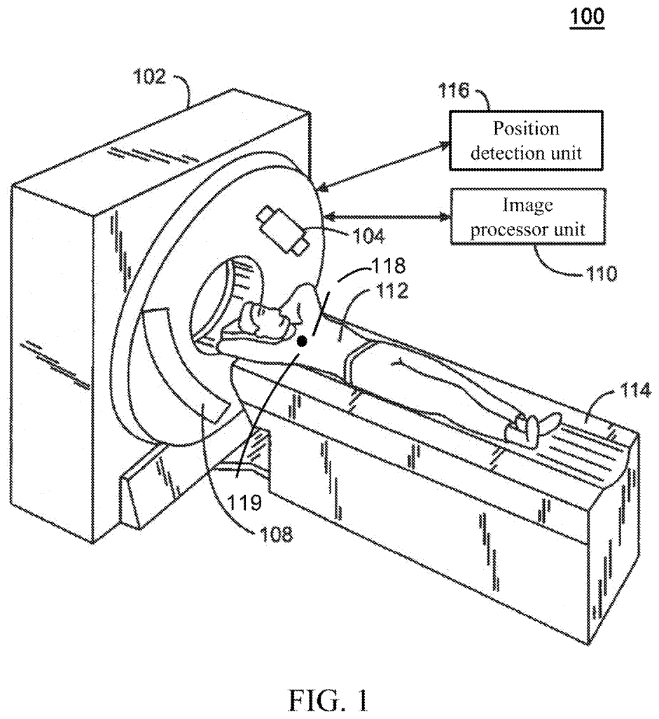

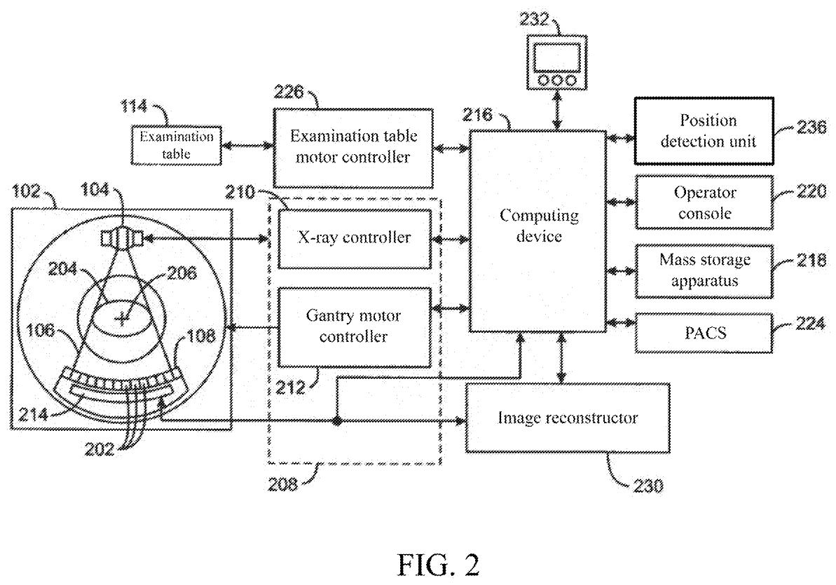

FIG. 1 shows an exemplary imaging CT imaging system 100 configured for CT imaging. Specifically, the CT imaging system 100 is configured to image a subject to be scanned 112 (such as a patient, an inanimate object, or one or more manufactured components) and/or a foreign object (such as an implant and/or a contrast agent present in the body). In one implementation, the CT imaging system 100 includes a gantry 102, which in turn may further include at least one X-ray source 104. The at least one X-ray source is configured to project an X-ray radiation beam 106 (see FIG. 2) for imaging the subject to be scanned 112 lying on an examination table 114. Specifically, the X-ray source 104 is configured to project the X-ray radiation beam 106 toward a detector array 108 positioned on the opposite side of the gantry 102. Although FIG. 1 depicts only one X-ray source 104, in certain implementations, a plurality of X-ray sources and detectors may be used to project a plurality of X-ray radiation beams 106, so as to acquire projection data corresponding to the patient at different energy levels. In some implementations, the X-ray source 104 may achieve dual-energy gemstone spectral imaging (GSI) by means of rapid peak kilovoltage (kVp) switching. In some implementations, the X-ray detectors which are used are photon counting detectors capable of distinguishing X-ray photons of different energies. In other implementations, dual-energy projections are generated using two sets of X-ray sources and detectors, wherein one set of X-ray sources and detectors is set to low kVp and the other set is set to high kVp. It should therefore be understood that the methods described herein may be implemented using single-energy acquisition techniques and dual-energy acquisition techniques.

The CT imaging system 100 may be used for CT imaging in a variety of scenarios. In one embodiment, the CT imaging system 100 may be used to image the position of an interventional object 118 in the body of the subject to be scanned 112 during a puncture procedure. Specifically, the CT imaging system 100 may perform CT imaging of the subject to be scanned 112 to generate a volumetric image, and identify the interventional object 118 in the volumetric image. On the basis of the identified interventional object 118, an operator (e.g., a doctor) may plan a puncture path so that the interventional object can accurately reach a predetermined target position. Further, the operator may perform operations such as sampling and drug administration.

In certain implementations, the CT imaging system 100 further includes an image processor unit 110 (e.g., a processor). In some examples, the image processor unit 110 may reconstruct, by means of using an iterative or analytical image reconstruction method, an image of a target volume or region of interest of the subject to be scanned 112. For example, the image processor unit 110 may reconstruct a volumetric image of the patient using an analytical image reconstruction method such as filtered back projection (FBP). As another example, the image processor unit 110 may reconstruct, by means of using an iterative image reconstruction method (such as advanced statistical iterative reconstruction (ASIR), conjugate gradient (CG), maximum likelihood expectation maximization (MLEM), model-based iterative reconstruction (MBIR), etc.), a volumetric image of the subject to be scanned 112. As further described herein, in some examples, in addition to the iterative image reconstruction method, the image processor unit 110 may use an analytical image reconstruction method (such as FBP).

In some other examples, the image processing unit 110 may identify the interventional object 118 in the volumetric image. The image processing unit 110 may identify the interventional object 118 according to brightness values of different pixels in the volumetric image. Generally speaking, compared with low-density objects such as muscles and bones of the subject to be scanned 112, the interventional object has higher density and therefore has stronger X-ray absorption, and correspondingly has a higher grayscale in the image. Accordingly, the image processing unit 110 may identify the interventional object 118 by means of a threshold algorithm.

In addition, the imaging system 100 may further include a position detection unit 116. The position detection unit 116 may be used to detect the position of the interventional object 118 relative to the subject to be scanned 112. Specifically, the position detection unit 116 may include an apparatus such as a 3D camera or a laser radar, which determines the position of the interventional object by detecting a part of the interventional object 118 exposed outside of the body of the subject to be scanned 112. The position detection unit 116 is further in communication with other parts of the imaging system 100 to send detected position information to the imaging system 100. In addition, the position detection unit 116 may further be a position sensor connected to the interventional object 118, and directly communicate with the imaging system 100. At said time, the position of the position detection unit 116 can represent the position of the interventional object 118. The function of the above position information will be described in detail below in the present application.

In some CT imaging system configurations, the X-ray source projects a conical X-ray radiation beam, which is collimated to be located within an X-Y-Z plane of a Cartesian coordinate system, and the plane is usually referred to as an “imaging plane”. The X-ray radiation beam passes through a subject being imaged, such as a patient or a subject to be scanned. The X-ray radiation beam is irradiated on a detector element array after being attenuated by the subject. The intensity of the attenuated X-ray radiation beam received at the detector array depends on the attenuation of the radiation beam by the subject. Each detector element of the array produces a separate electrical signal that is a measure of the X-ray beam attenuation at the detector position. Attenuation measurements from all detector elements are individually acquired to generate a transmission profile.

In some CT imaging systems, a gantry is used to rotate the X-ray source and the detector array in the imaging plane around a subject to be imaged so that the angle at which the radiation beam intersects the subject is constantly changing. A set of X-ray radiation attenuation measurement results (e.g., projection data) from the detector array at one gantry angle is referred to as a “view”. A “scan” of the subject includes a set of views made at different gantry angles or viewing angles during one rotation of the X-ray source and detectors. Therefore, as used herein, the term “view” is not limited to the use described above with respect to projection data from one gantry angle. The term “view” is used to mean one data acquisition when there are a plurality of data acquisitions from different angles (whether from a CT imaging system or any other imaging modality (including a modality to be developed), and combinations thereof).

Projection data is processed to reconstruct images corresponding to two-dimensional slices acquired by means of the subject, or in some examples in which the projection data includes a plurality of views or scans, reconstruct images corresponding to three-dimensional rendering of the subject. A method for reconstructing an image from a set of projection data is referred to as a filtered back projection technique. Transmission and emission tomography reconstruction techniques also include statistical iterative methods, such as maximum likelihood expectation maximization (MLEM) and ordered subset expectation reconstruction techniques, as well as iterative reconstruction techniques. The method converts an attenuation measurement from a scan into an integer referred to as a “CT number” or “Hounsfield unit”, which is used to control the brightness of a corresponding pixel on a display device.

In an “axial” scan, when the X-ray beam is rotated within the gantry, the CT examination table having the patient positioned thereon may be moved to a desired position, and is then kept stationary, thereby collecting data. A plurality of measurements from slices of the target volume may be reconstructed to form an image of the entire volume.

To reduce the total scan time, a “helical” scan may be performed. To perform the “helical” scan, the patient is moved when data of a specified number of slices is acquired. Such systems produce a single helix from helical scanning of a conical beam. The helix mapped out by the conical beam produces projection data according to which an image in each specified slice can be reconstructed.

As used herein, the phrase “reconstructing an image” is not intended to exclude an example of the present technique in which data representing an image is generated rather than a viewable image. Thus, as used herein, the term “image” broadly refers to both a viewable image and data representing a viewable image. However, many implementations generate (or are configured to generate) at least one viewable image.

FIG. 2 shows an exemplary imaging system 200. According to aspects of the present disclosure, the imaging system 200 is configured to image a patient or a subject to be scanned 204 (e.g., the subject to be scanned 112 of FIG. 1). In an implementation, the imaging system 200 includes the detector array 108 (see FIG. 1). The detector array 108 further includes a plurality of detector elements 202, which together sense the X-ray radiation beam 106 (see FIG. 2) passing through the subject to be scanned 204 (such as a patient) to acquire corresponding projection data. Therefore, in one implementation, the detector array 108 is fabricated in a multi-slice configuration including a plurality of rows of units or detector elements 202. In such a configuration, one or more additional rows of detector elements 202 are arranged in a parallel configuration for acquiring projection data.

In certain implementations, the imaging system 200 is configured to traverse different angular positions around the subject to be scanned 204 to acquire required projection data. Therefore, the gantry 102 and components mounted thereon can be configured to rotate about a center of rotation 206 to acquire projection data at different energy levels, for example. Alternatively, in implementations in which a projection angle with respect to the subject to be scanned 204 changes over time, the mounted components may be configured to move along a generally curved line rather than a segment of a circumference.

Therefore, when the X-ray source 104 and the detector array 108 rotate, the detector array 108 collects the data of the attenuated X-ray beam. The data collected by the detector array 108 is then subjected to pre-processing and calibration to adjust the data so as to represent a line integral of an attenuation coefficient of the scanned subject to be scanned 204. The processed data is generally referred to as a projection.

In some examples, an individual detector or detector element 202 in the detector array 108 may include a photon counting detector that registers interactions of individual photons into one or more energy bins. It should be understood that the methods described herein may also be implemented using an energy integration detector.

An acquired projection data set may be used for base material decomposition (BMD). During the BMD, the measured projection is converted to a set of material density projections. The material density projections may be reconstructed to form one pair or a set of material density maps or images (such as bone, soft tissue, and/or contrast agent maps) of each corresponding base material. The density maps or images may then be associated to form a volumetric image of a base material (e.g., bone, soft tissue, and/or a contrast agent) in an imaging volume.

Once reconstructed, a base material image produced by the imaging system 200 displays internal features of the subject to be scanned 204 represented by the densities of two base materials. The density images can be displayed to demonstrate the foregoing features. Such features may include a lesion, size, and shape of a particular anatomical structure or organ, and other features should be discernible in the image on the basis of the skill and knowledge of an individual practitioner. In an interventional procedure, the internal features can further include the orientation, interventional depth, etc., of an interventional object (not shown). By determining the orientation of the interventional object and the distance between the interventional object and the lesion, a doctor or physician is able to better adjust the strategy of the interventional procedure. In one embodiment, before the start of the interventional procedure, the doctor or physician will perform path planning for the interventional procedure in advance. Planning typically requires imaging of the lesion in advance. On the basis of the results of the imaging, a reasonable puncture path of the interventional object can be planned according to the position, size, etc., of the lesion, so as to prevent the interference of important organs and bones on the interventional object. In the intervening process, the imaging system 200 may further perform continuous or intermittent imaging of a site to be punctured, thereby promptly determining the position of the interventional object, and determining whether there is a deviation from the plan and whether an adjustment is required. The inventors are aware that an initial puncture position of the interventional object has been optimized in advance, and therefore, the volumetric image around the interventional object is suitable for identifying the interventional object due to less interference.

The imaging system 200 may further include a position detection unit 236, which may be configured as the position detection unit 116 in FIG. 1. The position detection unit 236 may include a variety of sensors for detecting the position of the interventional object. For example, the position detection unit 236 may include a sensor such as a 3D camera, a laser radar, an acceleration sensor, or a gyroscope. The position detection unit 236 communicates with a computing device such as a computing device processor, and sends the above position information to the processor for processing. A specific process will be described in detail below.

In one implementation, the imaging system 200 includes a control mechanism 208 to control movement of the components, such as the rotation of the gantry 102 and the operation of the X-ray source 104. In certain implementations, the control mechanism 208 further includes an X-ray controller 210, configured to provide power and timing signals to the X-ray source 104. Additionally, the control mechanism 208 includes a gantry motor controller 212, configured to control the rotational speed and/or position of the gantry 102 on the basis of imaging requirements.

In certain implementations, the control mechanism 208 further includes a data acquisition system (DAS) 214, configured to sample analog data received from the detector elements 202, and convert the analog data to a digital signal for subsequent processing. The DAS 214 may further be configured to selectively aggregate analog data from a subset of the detector elements 202 into a so-called macro detector, as described further herein. The data sampled and digitized by the DAS 214 is transmitted to a computer or computing device 216. In an example, the computing device 216 stores data in a storage device or mass storage apparatus 218. For example, the storage device 218 may include a hard disk drive, a floppy disk drive, a compact disc-read/write (CD-R/W) drive, a digital versatile disc (DVD) drive, a flash drive, and/or a solid-state storage drive.

Additionally, the computing device 216 provides commands and parameters to one or more of the DAS 214, the X-ray controller 210, and the gantry motor controller 212 to control system operations, such as data acquisition and/or processing. In certain implementations, the computing device 216 controls system operations on the basis of operator input. The computing device 216 receives the operator input via an operator console 220 that is operably coupled to the computing device 216, the operator input including, for example, commands and/or scan parameters. The operator console 220 may include a keyboard (not shown) or a touch screen to allow the operator to specify commands and/or scan parameters.

Although FIG. 2 shows only one operator console 220, more than one operator console may be coupled to the imaging system 200, for example, for inputting or outputting system parameters, requesting examination, mapping data, and/or viewing images. Moreover, in certain implementations, the imaging system 200 may be coupled to, for example, a plurality of displays, printers, workstations, and/or similar devices located locally or remotely within an institution or hospital or in a completely different location via one or more configurable wired and/or wireless networks (such as the Internet and/or a virtual private network, a wireless telephone network, a wireless local area network, a wired local area network, a wireless wide area network, a wired wide area network, etc.).

In one implementation, for example, the imaging system 200 includes or is coupled to a picture archiving and communication system (PACS) 224. In one exemplary implementation, the PACS 224 is further coupled to a remote system (such as a radiology information system or a hospital information system), and/or an internal or external network (not shown) to allow operators at different locations to supply commands and parameters and/or acquire access to image data.

The computing device 216 uses operator-supplied and/or system-defined commands and parameters to operate an examination table motor controller 226, which can in turn control the examination table 114. The examination table may be an electric examination table. Specifically, the examination table motor controller 226 may move the examination table 114 to properly position the subject to be scanned 204 in the gantry 102, so as to acquire projection data corresponding to a region of interest of the subject to be scanned 204.

As described previously, the DAS 214 samples and digitizes the projection data acquired by the detector elements 202. Subsequently, an image reconstructor 230 uses the sampled and digitized X-ray data to perform high-speed reconstruction. Although the image reconstructor 230 is shown as a separate entity in FIG. 2, in certain implementations, the image reconstructor 230 may form a part of the computing device 216. Alternatively, the image reconstructor 230 may not be present in the imaging system 200, and the computing device 216 may instead perform one or more functions of the image reconstructor 230. In addition, the image reconstructor 230 may be located locally or remotely and may be operably connected to the imaging system 200 by using a wired or wireless network. In some examples, computing resources in a “cloud” network cluster are available to the image reconstructor 230.

In one implementation, the image reconstructor 230 stores the reconstructed image in the storage device 218. Alternatively, the image reconstructor 230 may transmit the reconstructed image to the computing device 216 to generate usable patient information for diagnosis and evaluation. In certain implementations, the computing device 216 may transmit the reconstructed image and/or patient information to a display or display device 232, the display or display device being communicatively coupled to the computing device 216 and/or the image reconstructor 230. In some implementations, the reconstructed image may be transmitted from the computing device 216 or the image reconstructor 230 to the storage device 218 for short-term or long-term storage.

In some examples, the display 232 that is coupled to the computing device 216 may be used to display the interventional object and the volumetric image. The display 232 may also allow the operator to select a volume of interest (VOI) and/or request patient information, for example, via a graphical user interface (GUI), for subsequent scanning or processing. In some examples, the display 232 may be electrically coupled to the computing device 216, the CT imaging system 102, or any combination thereof. The computing device 216 may be located near the CT imaging system 102, or the computing device 216 may be located in another room, region, or remote location.

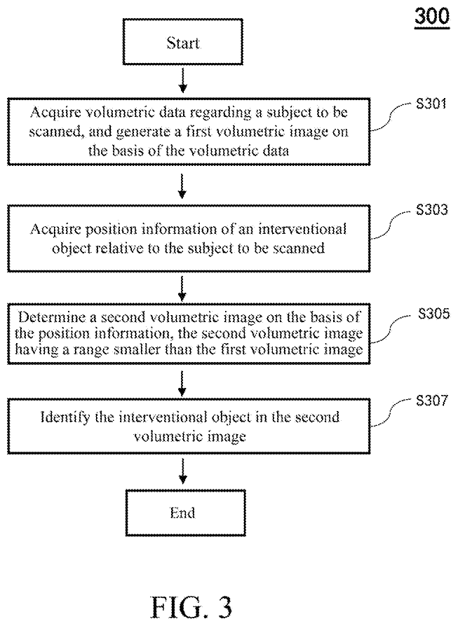

Various methods and processes described further herein (such as a method described below with reference to FIG. 3) may be stored as executable instructions in a non-transitory memory on a computing device (or a controller) in the imaging system 200. In one implementation, the examination table motor controller 226, the X-ray controller 210, the gantry motor controller 212, and the image reconstructor 230 may include such executable instructions in the non-transitory memory. In yet another implementation, the methods and processes described herein may be distributed on the CT imaging system 102 and the computing device 216.

As described herein, the accurate identification of the interventional object in the interventional procedure is very important. However, the inventors found that the degree of accuracy and the speed of identifying the interventional object are both affected in the volumetric image due to the interference of objects such as bones. To address at least part of the above problems, the present application proposes a series of improvements.

First, with reference to FIG. 3, a flowchart of a method 300 for identifying an interventional object in some embodiments of the present application is shown. It can be understood that the method can be implemented by the imaging system as set forth in any of the above embodiments.

In step 301, volumetric data regarding a subject to be scanned is acquired, and a first volumetric image is generated on the basis of the volumetric data. The step can be implemented by the imaging system described in any of the embodiments herein. For example, the step may be implemented by the processor of the imaging system 200. The means of acquiring the volumetric data and the means of generating the first volumetric image on the basis of the volumetric data may use the method described above in the present application, or may be any other means in the art, which will not be described herein again. The first volumetric image generated by the above step may have a large image range including the interventional object and a site to be scanned.

In step 303, position information of the interventional object relative to the subject to be scanned is acquired. The step may also be implemented by the processor of the imaging system 200. The position information obtained by detection is transmitted to the processor. Thus, the imaging system 200 can acquire a more specific position range of the site to be scanned including the interventional object. A method for detecting the position information of the interventional object relative to the subject to be scanned will be illustratively described in the following embodiments.

In the present application, the range of a second volumetric image is determined on the basis of the position information of the interventional object relative to the subject to be scanned. The position information of the interventional object relative to the subject to be scanned may be determined by the position detection unit 116 as set forth in the above embodiments. The above determination process may be implemented by the processor of the imaging system, and an exemplary description is given below.

The processor may receive a position detection signal from the position detection unit 116. It can be understood that the position detection unit 116 may communicate with the processor by any means, such as wired or wireless. The position detection unit 116 can detect the current position of the interventional object to produce the position detection signal and transmit same to the processor. The processor may determine the position information on the basis of the position detection signal, the position information including the position of a part of the interventional object exposed outside of the subject to be scanned relative to the subject to be scanned. The part of the interventional object exposed outside of the subject to be scanned is more easily detected, and the accuracy of detection is also higher than the part of the interventional object entering, by puncturing, the interior of the body of the subject to be scanned.

By means of the above scheme, the position information of the interventional object can be quickly detected. Moreover, since the above determination of the position information is performed on the basis of the interventional object exposed outside of the body of the subject to be scanned, the determination is more intuitive and does not rely on a medical imaging process.

Further, in step 305, a second volumetric image is determined on the basis of the position information, the second volumetric image having a range smaller than the first volumetric image. As described in step 303, by acquiring the position information of the interventional object relative to the subject to be scanned, the processor may obtain a more specific position range of the interventional object relative to the site to be scanned. On the basis of the foregoing, in step 305, the processor may further reduce the range of the above first volumetric image to obtain the second volumetric image. Since the above reduction is performed on the basis of the position information of the interventional object, the second volumetric image obtained through reduction can still include the interventional object, instead of excluding the interventional object.

On the basis that the position information of the interventional object relative to the subject to be scanned is determined, the second volumetric image may be determined on the basis of the position information. In some embodiments, the following process may be included: the processor may determine a position range of the interventional object in the first volumetric image on the basis of the above position information. Further, the range of the first volumetric image may be reduced on the basis of the position range of the interventional object to determine the second volumetric image, and the interventional object is included within the range of the second volumetric image.

In some embodiments, the orientation of the interventional object in the first volumetric image (e.g., a direction of extension of the above position information in the first volumetric image) may be determined according to the above position information. In some other embodiments, the possible position of the interventional object in a first volume space may be predicted according to the detection accuracy, i.e., error, of the above position information. Examples are not exhaustively enumerated.

With regards to volume, it should be noted that the first volumetric image and the second volumetric image are described herein, and in some examples, the two may be displayed by a display. However, in some other examples, this may not be the case. For example, considering that the first volumetric image includes more complete information of the site to be scanned, the first volumetric image may be displayed. In contrast, the second volumetric image may be a virtual concept, and the second volumetric image may be understood as a smaller range included in the first volumetric image and determined by the processor via step 305 described above, is used by the processor to perform subsequent processing, and is not separately displayed as an image.

Further, in step 307, the interventional object is identified in the second volumetric image. As set forth above, the second volumetric image is a smaller range in the first volumetric image. At this time, the processor may more efficiently and accurately identify the interventional object from the volumetric image having the smaller range. The means by which the interventional object is identified may vary depending on differences of imaging means. An exemplary description of a means for identifying an interventional object in CT imaging is given below, and is not intended to be an exclusive limitation. Under the teaching of the present disclosure, a person skilled in the art could make appropriate transformations.

In some embodiments, the interventional object may be identified by means of a threshold algorithm. A volumetric image obtained by CT imaging usually includes a plurality of pixels having different grayscales. The pixel grayscale value is related to the density of an object to be scanned. Specifically, if the density of the object to be scanned is high, the object to be scanned has high absorption of X-rays, and correspondingly, the grayscale value in a CT scanning image is also high. When the density of the object to be scanned is low, the object to be scanned has low absorption of X-rays, and the grayscale value in the CT scanning image is also low. During an interventional procedure, the interventional object has a higher density than the muscles and organs of the subject to be scanned, and therefore has a higher degree of X-ray absorption. The interventional object has a higher grayscale value in the CT scanning image. Accordingly, a grayscale value threshold may be set for filtering. The interventional object may be identified by filtering to retain pixels having high grayscale values and remove pixels having low grayscale values. It can be understood that the above description is merely an exemplary description of a threshold algorithm, and an actual threshold algorithm may be appropriately transformed under the teachings of the present disclosure.

In some other embodiments, the interventional object may be identified by means of an artificial neural network. The artificial neural network may be divided into two or more layers, such as an input layer for receiving an input image, an output layer for outputting an output image, and/or one or more intermediate layers. Layers of the neural network represent different groups or sets of artificial neurons, and may represent different functions that may be executed with respect to the input image (e.g., the second volumetric image) to identify an object (e.g. the interventional object) of the input image. The artificial neurons in the layers of the neural network may examine individual pixels in the input image. The artificial neurons use different weights in a function applied to the input image, so as to identify the object in the input image. The neural network produces the output image by assigning or associating different pixels in the output image with the interventional object on the basis of analysis of pixel characteristics. A method for training the artificial neural network may be arbitrary in the art, and will not be described herein again.

In conventional CT imaging, the site to be scanned typically includes a high-density object such as a bone. The difference between the grayscale value of the high-density object and the grayscale value of the interventional object is small in the volumetric image generated by scanning. The foregoing makes the processor susceptible to the interference of high-density objects such as bones when identifying the interventional object. At this time, multiple rounds of iterative calculations are usually required to eliminate the above interference. Identification in the above method usually takes a long time and is prone to misjudgment. In contrast, the method set forth in the above embodiments of the present application solves the above problems simply and efficiently. Specifically, in the present application, the position of the interventional object in the body of the subject to be scanned is determined by means of determining the positional relationship between the interventional object and the subject to be scanned. On the basis of the foregoing determination, identifying the interventional object in a smaller range (such as the second volumetric image) compared with an original volumetric image (such as the first volumetric image) can prevent the interference of high-density objects such as bones on the identification of the interventional object, and can also reduce the range of an image that needs to be identified, thereby improving the accuracy and speed of identifying the interventional object.

It should be noted that the order of the steps in the above method is not determined to be constant. In some embodiments, the generation of the first volumetric image may be performed before the determination of the position information. In some other embodiments, the determination of the position information may be performed before generating the first volumetric image. In addition, the determination of the position information and the generation of the first volumetric image may also be performed simultaneously. Examples are not exhaustively enumerated.

The inventors are aware that the relative position information between the interventional object and the subject to be scanned may have different spatial coordinates from the first volumetric image obtained by scanning by the imaging system, so it is difficult to directly obtain the prediction of the position of the interventional object in the first volumetric image according to the above position information. An exemplary description of how the position of the interventional object in the first volumetric image is determined on the basis of the position information is given below. In some embodiments, the spatial position of the subject to be scanned and the above first volumetric image may be registered so that the position information and the first volumetric image spatially have a correspondence. Further, on the basis of the registered position information, the position information of the interventional object in the first volumetric image may be predicted. In the method of the above embodiments of the present application, by means of establishing a spatial relationship of one-to-one correspondence between the position information (which may also be understood as real spatial coordinates of the interventional object and the subject to be scanned) and the first volumetric image, the two can directly correspond to each other, so that the position of the interventional object in the first volume space can be determined. It should be noted that the above registration process is not necessary for every scan. The registration may be performed only when the imaging system is mounted for the first time, and during subsequent scanning processes, since the position of the examination bed 114 where the subject to be scanned is located remains unchanged, no further registration is required. Of course, during a certain scan period, the above registration may also be calibrated to ensure the accuracy of registration.

An exemplary description of the registration process is given below with reference to FIG. 1. As shown in FIG. 1, the acquisition of volumetric data is formed by the detector array 108 acquiring the X-ray radiation beam 106 emitted by the X-ray source 104, and the volumetric data is processed to obtain a volumetric image. In contrast, the spatial information of the subject to be scanned (or the interventional object) is acquired by the position detection unit 116. Since the volumetric image and spatial information of the subject to be scanned are obtained from different routes, there may be a situation in which the two do not correspond spatially. As an exemplary illustration, a common reference point 119 may be defined for the two. As shown in FIG. 1, the position of the reference point 119 may be arbitrary. For example, the position may be a certain fixed position above the examination bed 114. Further, the reference point 119 may be used as both the volumetric image of the subject to be scanned and the spatial coordinate origin of the spatial information of the subject to be scanned. On the basis of the foregoing, the registration of the spatial position and the volumetric image of the subject to be scanned is achieved. Correspondingly, the spatial position of the interventional object 118 and the position thereof in the volumetric image are also registered. The above registration means is merely one example of the present application. Under the teaching of this example, a person skilled in the art could further use other appropriate means to perform registration, which will not be described herein again.

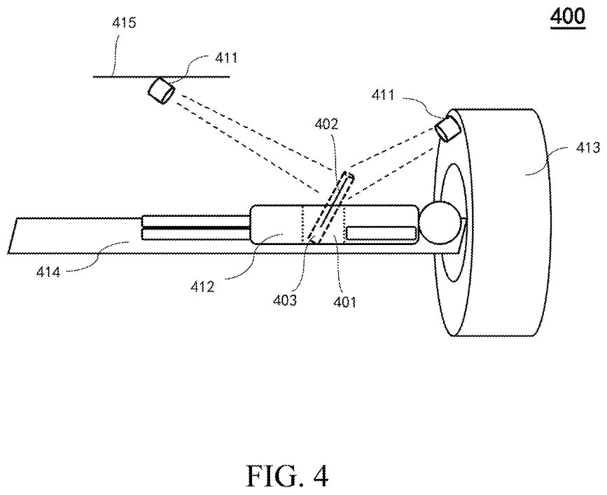

The reduction of a volumetric image after the registration of the interventional object and the volumetric image will be further described in detail below with reference to FIG. 4. As shown in FIG. 4, a schematic diagram 400 of determining a position range of an interventional object in a first volumetric image in some embodiments of the present application is shown. The configuration of a gantry 413 and an examination table 414, and the specific means of scanning a subject to be scanned 412 to acquire a volumetric image of a site to be scanned thereof, may be as described in FIGS. 1 and 2 and any corresponding embodiments thereof herein, and will not be described again. By using a volumetric data acquisition apparatus (not shown, e.g., an X-ray emission apparatus and components thereof such as detectors) within the gantry 413, volumetric data of the site to be scanned 401 may be obtained, and the volumetric data may be further processed to obtain a first volumetric image (not shown). It can be understood that the first volumetric image corresponds to the site to be scanned 401. Position information of an interventional object 402 may be determined by using identification of the interventional object 402 by a position detection unit 411.

The position range of the interventional object 402 in the first volumetric image (not shown) may be determined on the basis of the position information of the interventional object 402. Specifically, a spatial range 403 including the interventional object 402 may be determined on the basis of the position information. Further, as shown in FIG. 4, the first volumetric image (which corresponds to the site to be scanned 401) and the spatial range 403 are registered so that the two are at least partially coincident. A part in which the first volumetric image is coincident with the spatial range 403 may be determined to be the position range of the interventional object 402 in the first volumetric image. On the basis of the foregoing position range, a processor of an imaging system may reduce the first volumetric image to obtain a second volumetric image, and perform identification of the interventional object 402.

The size of the above spatial range 403 may be considered according to a variety of factors. The above spatial range 403 may be a certain spatial range including the interventional object 402. For example, the spatial range 403 may be a certain range taking into account a detection error of the position detection unit 411, and/or at least one of various factors such as the movement of the detection bed 414, advancement of the interventional object 402, and slight displacement of the subject to be scanned 412 during use of the imaging system. Examples are not exhaustively enumerated in the present application. Such a configuration means that the error of the position detection of the interventional object 402 can be sufficiently considered, and the range of the first volumetric image can be reduced.

The position detection unit 411 may have a variety of configurations. In one example, the position detection unit 411 may be any one of a 3D camera and a laser radar. The above apparatus may be mounted at a suitable position, for example, directly above the imaging system. The above apparatus may acquire image data in an environment and identify, from the image data, the interventional object exposed outside of the body of the subject to be scanned. The mounted position detection unit 411 has a fixed position, thereby being capable of ensuring that the spatial information and the position of the volumetric image can correspond to each other after one registration. In one example, one position detection unit 411 is configured. The position detection unit 411 may be mounted on a top surface 415 as shown in FIG. 4. Alternatively, the position detection unit 411 is mounted at the top of the gantry 413. In another example, a plurality of position detection units 411 can be included. The plurality of position detection units 411 are mounted at different positions, respectively, thereby facilitating more precise detection of the position information of the interventional object 402.

In addition, in another example, the position detection unit 411 may further be a position sensor (not shown) that is connected to the interventional object. The position sensor may be diverse, for example, an acceleration sensor and other conventional position sensors in the art. The position sensor may be configured to communicate with the imaging system, thereby determining the relative positional relationship thereof with the imaging system, and then registering with the volumetric image. The type of the position sensor may also be a combination of any of the above sensors to improve the detection accuracy, and examples are not exhaustively enumerated.

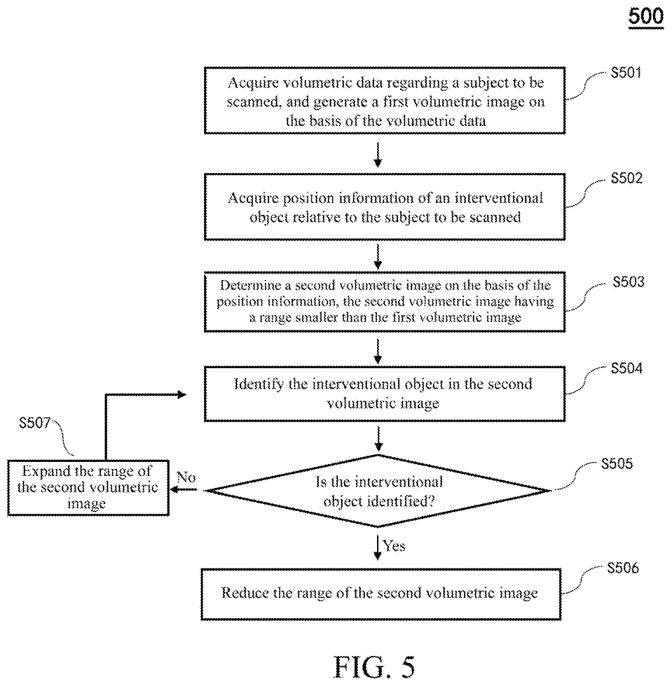

As described above in the present application, in the interventional procedure, the identification process of the interventional object might be continuously carried out as the interventional procedure progresses. That is, the operator needs to continuously identify (i.e., track) the position of the interventional object in the body of the subject to be scanned. The foregoing requires multiple imaging instances. Multiple imaging instances inevitably result in longer exposure of the operator and the subject to be scanned to an imaging environment such as X-rays. The inventors of the present application are aware that it is of great significance to improve accuracy and efficiency in the process of tracking the interventional object. With reference to FIG. 5, a flowchart 500 of a method for identifying an interventional object in some other embodiments of the present application is shown.

In step 501, volumetric data regarding a subject to be scanned is acquired, and a first volumetric image is generated on the basis of the volumetric data. The step can be implemented by the imaging system described in any of the embodiments herein. For example, the step may be implemented by the processor of the imaging system 200. The first volumetric image generated by the above step may have a large image range including an interventional object and a site to be scanned.

In step 502, position information of the interventional object relative to the subject to be scanned is acquired. The step may also be implemented by the processor of the imaging system 200. The position information obtained by detection is transmitted to the processor. Thus, the imaging system 200 can acquire a more specific position range of the site to be scanned including the interventional object.

In step 503, a second volumetric image is determined on the basis of the position information, the second volumetric image having a range smaller than the first volumetric image. The processor may further reduce the range of the above first volumetric image to obtain the second volumetric image. Since the above reduction is performed on the basis of the position information of the interventional object, the second volumetric image obtained through reduction can still include the interventional object, instead of excluding the interventional object.

In step 504, the interventional object is identified in the second volumetric image. As set forth above, the second volumetric image is a smaller range in the first volumetric image. At this time, the processor may more efficiently and accurately identify the interventional object from the volumetric image having the smaller range.

It can be understood that each of steps 501 to 504 may reference steps 301 to 307 described above in the present application, respectively, and may also be subjected to appropriate adjustments.

Further, in step 505, it is determined whether the interventional object is identified. Moreover, the range of the second volumetric image is adjusted on the basis of the identification result. The inventors are aware that there may be a deviation in the above identification result, or that there is room for adjustment in the range of the second volumetric image. On the basis of the foregoing, in step 505, by appropriately adjusting the range of the second volumetric image on the basis of the identification result, the degree of accuracy and efficiency of identifying the interventional object can be further increased.

Specifically, in step 506, in response to the interventional object being identified, the range of the second volumetric image is reduced, wherein the reducing is substantially performed taking the interventional object as the center. When the processor identifies the interventional object in the second volumetric image, it is demonstrated that the determination result of the current second volumetric image is accurate. Further, the range of the second volumetric image may be further reduced to increase the efficiency in the subsequent process of identifying and tracking the interventional object. The reduction may be substantially performed taking the interventional object as the center. It can be understood that the interventional object is typically needle-shaped, and the path of travel thereof is also generally rectilinear and thus has a fixed orientation. At this time, reducing the range of the second volumetric image taking the interventional object as the center can prevent as much as possible the exclusion of the interventional object in the reduced volumetric image due to the reduction. The meaning of “substantially” is to allow a certain deviation in the above reduction.

According to an embodiment of another aspect, in step 507, the range of the second volumetric image may be expanded in response to the interventional object being unidentified, and the interventional object is identified in the expanded second volumetric image. Considering, for example, the detection error of the position detection unit, the second volumetric image obtained through reduction may not include the interventional object. At this time, the range of the second volumetric image may be expanded. In addition, in other embodiments, part of the interventional object may be unidentified. For example, the tip of the interventional object is unidentified, which may also adversely affect the imaging guidance of the interventional procedure. At this time, the range of the second volumetric image may likewise be expanded. In one embodiment, the expanded volumetric image range may be set by a machine, for example, may be preset according to a possible position detection unit error. In another embodiment, the identification of the interventional object may be expanded to the entire first volumetric image range.

It can be understood that steps 506 and 507 described above are merely exemplary illustrations of the adjustment set forth in step 505. Under the teachings of the present application, the adjustment may also be combined. For example, using the method disclosed in step 507, the interventional object is identified by means of expanding the range of the second volumetric image, and then, taking the interventional object as the center, the range of the second volumetric image is reduced by using the method disclosed in step 506, thereby increasing the identification efficiency. For another example, using the method disclosed in step 507, the interventional object cannot be successfully identified by means of expanding the range of the second volumetric image, and then, the method of step 507 may be continuously used for multiple iterations to ensure that the interventional object is finally identified. Examples are not exhaustively enumerated.

By means of the above method, even during the process of a continuous interventional procedure and multiple imaging instances, the accuracy and efficiency of the identification of the interventional object can be dynamically improved, and continuous tracking imaging of the interventional object that is constantly changing position can be adaptively performed.

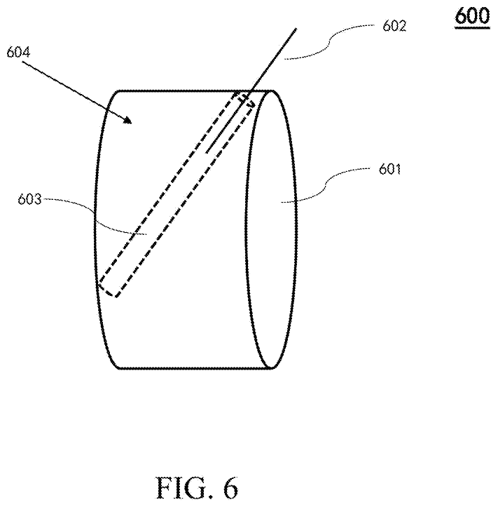

The interventional object identification process of the present application will be further described in detail with reference to FIG. 6. A schematic diagram 600 of identifying an interventional object in some embodiments of the present application is shown in FIG. 6. A first volumetric image 601 may be obtained by the imaging system 100 by the means described in any of the above embodiments. The interventional object 602 at least partially punctures the body of a subject to be scanned (not shown) in the interventional procedure. As set forth in the above embodiments herein, position information of the interventional object 602 relative to the subject to be scanned is acquired by the imaging system 100 for determining a second volumetric image 603. It can be understood that the second volumetric image 603 may be virtual and not used for displaying. As can be seen from FIG. 6, the range of the second volumetric image 603 is significantly smaller than the first volumetric image 601, and the second volumetric image 603 is suitable for quickly and accurately identifying the interventional object 602.

As set forth above in the present application, the range of the second volumetric image 603 may further be constantly adjusted in the continuous tracking process of the interventional procedure. In one embodiment, the initial range of the second volumetric image 603 may be preset according to the detection accuracy (or tolerance) of a position detection unit. Further, according to the identification result, the imaging system can expand or reduce the range of the second volumetric image 603, further increasing the identification efficiency, and facilitating tracking of the interventional object.

The present application further provides embodiments that facilitate the interventional procedure by an operator. In some embodiments, after the interventional object 602 is identified in the second volumetric image 603, the first volumetric image 601 and the identified interventional object 602 may be displayed. The above display may be implemented by the display 232. By means of the above display process, the operator can promptly grasp the position of the interventional object in the body of the subject to be scanned, so that the next operation can be accurately determined.

In some other embodiments, the imaging system 100 may further adjust the angle of the first volumetric image 601 on the basis of the identified interventional object 602. In the adjustment, an angle that facilitates viewing by the operator may be automatically selected on the basis of the orientation of the interventional object 602 to adjust the angle of the first volumetric image 601 (e.g., to be adjusted to the viewing angle of 604 shown in FIG. 6), so that the operator can be automatically assisted in performing the interventional procedure.

In some embodiments of the present application, an imaging system is further provided, including a volumetric data acquisition apparatus, for acquiring volumetric data regarding a subject to be scanned, a processor, configured to perform the method as set forth in any of the above embodiments of the present application, and a display, for receiving a signal from the processor so as to carry out display. The imaging system may be the imaging system 100, the imaging system 200, or any imaging system as set forth in the present application. The volumetric data acquisition apparatus may be the data acquisition system 214, etc., as set forth in the present application. The display may be the display 232 as set forth in the present application. Examples are not exhaustively enumerated.

Some embodiments of the present application further provide a non-transitory computer-readable medium, having a computer program stored therein, the computer program having at least one code segment, and the at least one code segment being executable by a machine so as to enable the machine to perform the steps of the method in any of the embodiments described above.

Correspondingly, the present disclosure may be implemented as hardware, software, or a combination of hardware and software. The present disclosure may be implemented in at least one computer system by using a centralized means or a distributed means, different elements in the distributed means being distributed on a number of interconnected computer systems. Any type of computer system or other device suitable for implementing the methods described herein is considered to be appropriate.

The various embodiments may also be embedded in a computer program product, which includes all features capable of implementing the methods described herein, and the computer program product is capable of executing these methods when loaded into a computer system. A computer program in this context means any expression in any language, code, or symbol of an instruction set intended to enable a system having information processing capabilities to execute a specific function directly or after any or both of a) conversion into another language, code, or symbol; and b) duplication in a different material form.

The purpose of providing the above specific embodiments is to allow the disclosure of the present application to be understood more thoroughly and comprehensively; however, the present application is not limited to said specific embodiments. A person skilled in the art should understand that various modifications, equivalent replacements, changes and the like can be further made to the present application and should be included in the scope of protection of the present application as long as these changes do not depart from the spirit of the present application.

What is claimed is:

1. A method for identifying an interventional object, comprising:
acquiring volumetric data regarding a subject to be scanned, and generating a first volumetric image on the basis of the volumetric data;
acquiring position information of the interventional object relative to the subject to be scanned;
determining a second volumetric image on the basis of the position information, the second volumetric image having a range smaller than the first volumetric image; and
identifying the interventional object in the second volumetric image.

2. The method according to claim 1, wherein the acquiring position information of the interventional object relative to the subject to be scanned comprises:
receiving a position detection signal from a position detection unit; and
determining the position information on the basis of the position detection signal, the position information comprising the position of a part of the interventional object exposed outside of the subject to be scanned relative to the subject to be scanned.

3. The method according to claim 1, wherein the determining a second volumetric image on the basis of the position information comprises:
determining a position range of the interventional object in the first volumetric image on the basis of the position information; and
reducing the range of the first volumetric image on the basis of the position range of the interventional object so as to determine the second volumetric image, the interventional object being comprised in the range of the second volumetric image.

4. The method according to claim 3, wherein the determining a position range of the interventional object in the first volumetric image on the basis of the position information comprises:
determining a spatial range comprising the interventional object on the basis of the position information;
registering the first volumetric image and the spatial range so that the two are at least partially coincident; and
determining a part in which the first volumetric image is coincident with the spatial range as the position range of the interventional object in the first volumetric image.

5. The method according to claim 1, further comprising:
adjusting the range of the second volumetric image on the basis of the identification result.

6. The method according to claim 5, wherein the adjusting comprises:
expanding the range of the second volumetric image in response to the interventional object being unidentified or partially unidentified; and
identifying the interventional object in the expanded second volumetric image.

7. The method according to claim 5, wherein the adjusting comprises:
reducing the range of the second volumetric image in response to the interventional object being identified, the reducing being substantially performed taking the interventional object as a center.

8. The method according to claim 1, further comprising:
displaying the first volumetric image and the identified interventional object.

9. The method according to claim 1, further comprising:
adjusting an angle of the first volumetric image on the basis of the identified interventional object.

10. The method according to claim 1, wherein the first volumetric image comprises at least one of a magnetic resonance image and a computed tomography image.

11. The method according to claim 2, wherein the position detection unit comprises at least one of the following:
a 3D camera, a laser radar, and a position sensor connected to the interventional object.

12. An imaging system, comprising:
a volumetric data acquisition apparatus, for acquiring volumetric data regarding a subject to be scanned;
a processor, configured to perform the method according to claim 1; and
a display, for receiving a signal from the processor so as to carry out display.

13. The system according to claim 12, further comprising:
a position detection unit, for detecting the position of an interventional object relative to the subject to be scanned so as to generate a position detection signal.

14. The system according to claim 13, wherein the position detection unit comprises at least one of the following:
a 3D camera, a laser radar, and a position sensor connected to the interventional object.

15. A non-transitory computer-readable medium, having a computer program stored thereon, the computer program having at least one code segment, and the at least one code segment being executable by a machine so as to enable the machine to perform steps of the method according to claim 1.
